(19) United States
(12) Patent Application Publication
(10) Pub. No.: US 2013/0116566 A1
(43) Pub. Date: May 9, 2013

(54) ULTRASONIC DIAGNOSTIC SYSTEM AND ULTRASONIC DIAGNOSTIC METHOD

(71) Applicants: Takeshi SATO, Nasushiobara-shi (JP); Shinichi HASHIMOTO, Otawara-shi (JP); Kenji HAMADA, Otawara-shi (JP)

(72) Inventors: Takeshi SATO, Nasushiobara-shi (JP); Shinichi HASHIMOTO, Otawara-shi (JP); Kenji HAMADA, Otawara-shi (JP)

(21) Appl. No.: 13/656,984

(22) Filed: Oct. 22, 2012

(30) Foreign Application Priority Data
Nov. 9, 2011 (JP) 2011-245910
Sep. 19, 2012 (JP) 2012-205376

(51) Int. Cl. A61B 8/14

(52) U.S. Cl. 600/447

(57) ABSTRACT

According to one embodiment, an ultrasonic diagnostic system includes a data acquiring unit, a beam forming processing unit, a processor and an output unit. The data acquiring unit is configured to acquire reception signals corresponding to ultrasonic transducers by transmitting and receiving ultrasonic waves to and from an object using the ultrasonic transducers. The beam forming processing unit is configured to apply beam forming to the reception signals. The processor is configured to generate ultrasonic image data based on reception signals subjected to the beam forming. The output unit is configured to output the reception signals before the beam forming to an outside terminal.

### CROSS REFERENCE TO RELATED APPLICATION

This application is based upon and claims the benefit of priority from Japanese Patent Application No. 2011-245910, filed Nov. 9, 2011 and Japanese Patent Application No. 2012-205376, filed Sep. 19, 2012; the entire contents of Japanese Patent Application No. 2011-245910 and Japanese Patent Application No. 2012-205376 are incorporated herein by reference.

### FIELD

Embodiments described herein relate generally to an ultrasonic diagnostic system and an ultrasonic diagnostic method.

### BACKGROUND

In recent years, a mobile ultrasonic diagnostic apparatus capable of using with holding in hands begins to become common. However, a drive voltage of an ultrasonic transmission system included in an ultrasonic diagnostic apparatus is relatively large. For example, drive voltages of a high pressure SW, a transmission/reception separation circuit and a transmission circuit included in the transmission system are over 100V. Therefore, it is difficult to increase a density of an IC (integrated circuit) on the transmission system in the ultrasonic diagnostic apparatus because of securing a sufficient withstanding pressure.

Therefore, conventionally, downsizing and price reduction for the mobile ultrasonic diagnostic apparatus are attempted by reducing the number of transmission channels. However, there is a problem that high image quality cannot be obtained by the conventional mobile ultrasonic diagnostic apparatus since the number of the transmission channels is restricted. Specifically, as described above, it is difficult to increase the number of the transmission channels because of securing a sufficient withstanding pressure since a drive voltage of the transmission system in the ultrasonic diagnostic apparatus is high. Therefore, for example, a linear electron array probe which includes more than 100 ultrasonic transducers cannot be connected with the conventional mobile ultrasonic diagnostic apparatus.

On the contrary, when the number of the transmission channels is tried to increase, a circuit size of the transmission system becomes large and downsizing becomes difficult. Specifically, there is a problem that the circuit size of the transmission system becomes large depending on the number of transmission channels since a small and low-cost IC for a low voltage cannot be used for the circuit of the transmission system.

It is an object of the present invention to provide a smaller ultrasonic diagnostic system and an ultrasonic diagnostic method which can obtain an ultrasonic diagnostic image with a higher image quality.

### BRIEF DESCRIPTION OF THE DRAWINGS

In the accompanying drawings:

FIG. 1 is a block diagram of an ultrasonic diagnostic system according to the first embodiment of the present invention;

FIG. 2 is a diagram to show switching states of the reception signal output by the output selection SW shown in FIG. 1;

FIG. 3 is a functional block diagram of the DSP shown in FIG. 1;

FIG. 4 is a diagram describing a correction method of the reception delay times in the delay time correction part shown in FIG. 1; and

FIG. 5 is a block diagram showing an ultrasonic diagnostic system according to the second embodiment of the present invention.

### DETAILED DESCRIPTION

In general, according to one embodiment, an ultrasonic diagnostic system includes a data acquiring unit, a beam forming processing unit, a processor and an output unit. The data acquiring unit is configured to acquire reception signals corresponding to ultrasonic transducers by transmitting and receiving ultrasonic waves to and from an object using the ultrasonic transducers. The beam forming processing unit is configured to apply beam forming to the reception signals. The processor is configured to generate ultrasonic image data based on reception signals subjected to the beam forming. The output unit is configured to output the reception signals before the beam forming to an outside terminal.

Further, according to another embodiment, an ultrasonic diagnostic system includes an ultrasonic diagnostic apparatus and a computer. The computer is connected to the ultrasonic diagnostic apparatus through a network. The ultrasonic diagnostic apparatus includes a data acquiring unit, a beam forming processing unit, a processor and an output unit. The data acquiring unit is configured to acquire reception signals corresponding to ultrasonic transducers by transmitting and receiving ultrasonic waves to and from an object using the ultrasonic transducers. The beam forming processing unit is configured to apply a first beam forming to the reception signals. The processor is configured to generate first ultrasonic image data based on reception signals subjected to the first beam forming. The output unit is configured to output the reception signals before the first beam forming to the computer. The computer functions as a data generation unit. The data generation unit is configured to apply a second beam forming to the reception signals before the first beam forming output from the output unit to generate second ultrasonic image data having a data size larger than that of the first ultrasonic image data based on the reception signals subjected to the second beam forming.

Further, according to another embodiment, an ultrasonic diagnostic system includes an ultrasonic diagnostic apparatus, a computer and an ultrasonic diagnostic image server. The ultrasonic diagnostic apparatus is placed in a medical institution. The computer is placed in the medical institution and has a display unit. The ultrasonic diagnostic image server is placed in a center side and connected with each of the ultrasonic diagnostic apparatus and the computer through a network. The ultrasonic diagnostic apparatus includes a data acquiring unit, a beam forming processing unit, a processor and an output unit. The data acquiring unit is configured to acquire reception signals corresponding to ultrasonic transducers by transmitting and receiving ultrasonic waves to and from an object using the ultrasonic transducers. The beam forming processing unit is configured to apply a first beam forming to the reception signals. The processor is configured to generate first ultrasonic image data based on reception signals subjected to the first beam forming. The output unit is configured to transmit the reception signals before the first beam forming to the ultrasonic diagnostic image server. The ultrasonic diagnostic image server includes a data generation unit and a data transmission unit. The data generation unit is configured to apply the second beam forming to the reception signals before the first beam forming output from the output unit to generate second ultrasonic image data having a data size larger than that of the first ultrasonic image data based on the reception signals subjected to the second beam forming. The data transmission unit is configured to transmit the second ultrasonic image data to the computer.

Further, according to another embodiment, an ultrasonic diagnostic system includes a data reception unit and a data generation unit. The data reception unit is configured to receive reception signals before a beam forming and corresponding to ultrasonic transducers from an ultrasonic diagnostic apparatus through a network. The reception signals are acquired by transmitting and receiving ultrasonic waves to and from an object using the ultrasonic transducers. The data generation unit is configured to apply a beam forming to the reception signals to generate ultrasonic image data based on reception signals subjected to the beam forming.

Further, according to another embodiment, an ultrasonic diagnostic system includes a data reception unit, a data generation unit and a data transmission unit. The data reception unit is configured to receive reception signals before a beam forming and corresponding to ultrasonic transducers from an ultrasonic diagnostic apparatus through a network. The reception signals are acquired by transmitting and receiving ultrasonic waves to and from an object using the ultrasonic transducers. The data generation unit is configured to apply a beam forming to the reception signals to generate ultrasonic image data based on reception signals subjected to the beam forming. The data transmission unit is configured to transmit the ultrasonic image data to a computer having a display unit through a network.

Further, according to another embodiment, an ultrasonic diagnostic method includes: acquiring reception signals corresponding to ultrasonic transducers by transmitting and receiving ultrasonic waves to and from an object using the ultrasonic transducers; applying beam forming to the reception signals; generating ultrasonic image data with a processor based on reception signals subjected to the beam forming; and outputting the reception signals before the beam forming to an outside terminal.

An ultrasonic diagnostic system and an ultrasonic diagnostic method according to an embodiment of the present invention will be described with reference to the accompanying drawings.

### First Embodiment

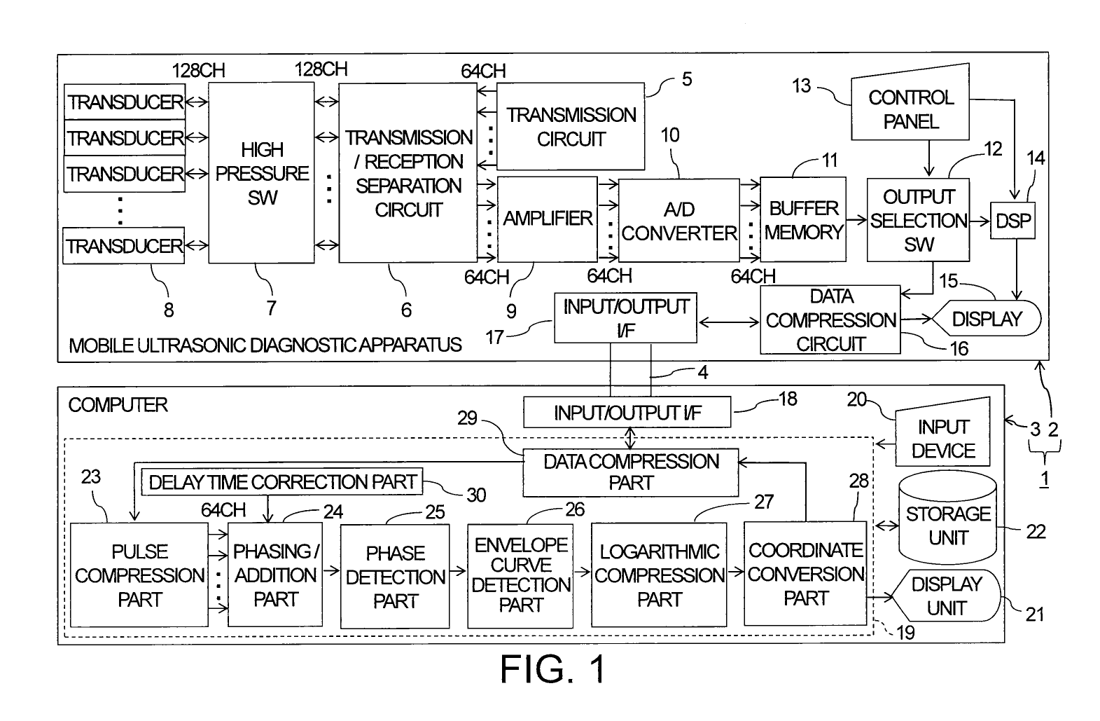

FIG. 1 is a block diagram of an ultrasonic diagnostic system according to the first embodiment of the present invention.

An ultrasonic diagnostic system 1 is configured by connecting a mobile ultrasonic diagnostic apparatus 2 with a computer 3 with a transmission cable 4. The mobile ultrasonic diagnostic apparatus 2 includes a transmission circuit 5, a transmission/reception separation circuit 6, a high pressure SW 7, multiple ultrasonic transducers 8, an amplifier 9, an A/D (analog to digital) converter 10, a buffer memory 11, an output selection SW 12, a control panel 13, a digital signal processor (DSP) 14, a display 15, a data compression circuit 16 and an input/output interface (I/F) 17.

The ultrasonic transducers 8 are connected with multiple transmission channels and reception channels via the transmission/reception separation circuit 6 and the high pressure SW 7. Each ultrasonic transducer 8 has a function to convert a transmission signal applied as an electrical signal from the transmission circuit 5 via the transmission/reception separation circuit 6 and the high pressure SW 7 into an ultrasonic transmission signal to transmit to an object Each ultrasonic transducer 8 also has a function to receive an ultrasonic reflected signal generated in the object by transmitting the ultrasonic signal, convert the ultrasonic reflected signal to an electrical reception signal and output the electrical reception signal to a reception channel.

Further, the multiple ultrasonic transducers 8 forms an ultrasonic probe. An arbitrary type of probe such as a convex type, a linear type or a sector type can be used as the ultrasonic probe.

The transmission circuit 5 is a circuit to generate a transmission signal for each transmission channel to output the transmission signal to the transmission/reception separation circuit 6. In the transmission circuit 5, a delay time is given to each transmission signal for giving directionality to respective ultrasonic signals transmitted from the multiple ultrasonic transducers 8 to form an ultrasonic transmission beam. Then, the multiple transmission signals generated in the transmission circuit 5 are output to the corresponding transmission channels respectively and applied to the respective ultrasonic transducers 8 via the transmission/reception separation circuit 6 and the high pressure SW 7.

The transmission/reception separation circuit 6 is a circuit to separate transmission signals, applied to the ultrasonic transducers 8 from the transmission circuit 5 via the high pressure SW 7, from reception signals, output from the ultrasonic transducers 8 via the high pressure SW 7. Specifically, the transmission/reception separation circuit 6 applies transmission signals, received from the transmission circuit 5, to the ultrasonic transducers 8 via the high pressure SW 7 and outputs reception signals, acquired from the ultrasonic transducers 8 via the high pressure SW 7, to the amplifier 9.

The high pressure SW 7 is a switch to convert signal paths for applying transmission signals, output from the transmission/reception separation circuit 6, to the ultrasonic transducers 8 and signal paths for outputting reception signals, output from the ultrasonic transducers 8, to the transmission/reception separation circuit 6.

It is preferable that a signal, to which pulse compression can be performed, such as a chirp wave having a low peak voltage is used as a transmission signal generated in the transmission circuit 5 and applied to the ultrasonic transducer 8 from the perspective of decreasing a drive voltage of the transmission circuit 5 and acquiring a sufficient sensitivity. A pulse compression technique is one of techniques which allow driving the transmission circuit 5 with a low voltage about 20V to acquire a reception sensitivity equivalent to that when the transmission circuit 5 is driven with a high voltage. Further, the chirp wave is a wave derived by changing a frequency of a sine wave with time.

Especially, using a chirp wave having a Gauss envelope curve as a transmission signal makes it possible to reduce a peak voltage of the transmission signal to use an IC with a high integration for a low voltage. Additionally, a pulse compression processing of a reception signal received corresponding to a transmission signal consisting of a chirp wave allows acquiring a sensitivity equivalent to that when a reception signal having a pulse waveform including the Gauss envelope curve having a similar amplitude characteristic is received.

Therefore, it becomes possible to integrate circuits used in the transmission system of the mobile ultrasonic diagnostic apparatus 2. Specifically, it becomes possible to configure the high pressure SW 7, the transmission/reception separation circuit 6 and the transmission circuit 5 with highly integrated IC. Further, more than 4 times as many channels as conventional channels can be equipped on a single IC chip. Consequently, the number of channels can be increased with downsizing circuits used in the transmission system of the mobile ultrasonic diagnostic apparatus 2.

As an example, the hand-held small mobile ultrasonic diagnostic apparatus 2 of approximately 80 mm×59 mm×25 mm can mount 64 transmission channels and reception channels respectively as shown in FIG. 1. Therefore, in the example shown in FIG. 1, the ultrasonic transducers 8 for 128 channels corresponding to 64 transmission channels and 64 reception channels are mounted.

On the other hand, the amplifier 9 which configures the reception system is a device to amplify the reception signals acquired by the respective reception channels to output the reception signals to the A/D converter 10.

The A/D converter 10 is a circuit to convert the analog reception signals for the respective reception channels output from the amplifier 9 to the digital reception signals. The multiple radio frequency (RF) reception signals, after A/D conversion, corresponding to the multiple ultrasonic transducers 8 are stored in the buffer memory 11.

Therefore, in the example shown in FIG. 1, the transmission circuit 5, the transmission/reception separation circuit 6, the high pressure SW 7, the multiple ultrasonic transducers 8, the amplifier 9, the A/D converter 10 and the buffer memory 11 function as a data acquisition unit which acquires multiple reception signals corresponding to the multiple ultrasonic transducers 8 by transmitting and receiving ultrasonic waves to and from an object with the multiple ultrasonic transducers 8. As long as the equivalent function can be provided, the data acquisition unit of the mobile ultrasonic diagnostic apparatus 2 may be configured by other elements.

The output selection SW 12 is a switch to select the output of reception signals, for respective reception channels, stored in the buffer memory 11 by operation of the control panel 13. It is possible to select either one or both of the DSP 14 and the computer 3 as the output or the outputs of the reception signals. When the computer 3 is the output, multiple RF signals corresponding to the multiple ultrasonic transducers 8 and the multiple reception channels are transmitted to the computer 3 through the transmission cable 4 via the data compression circuit 16 and the input/output I/F 17.

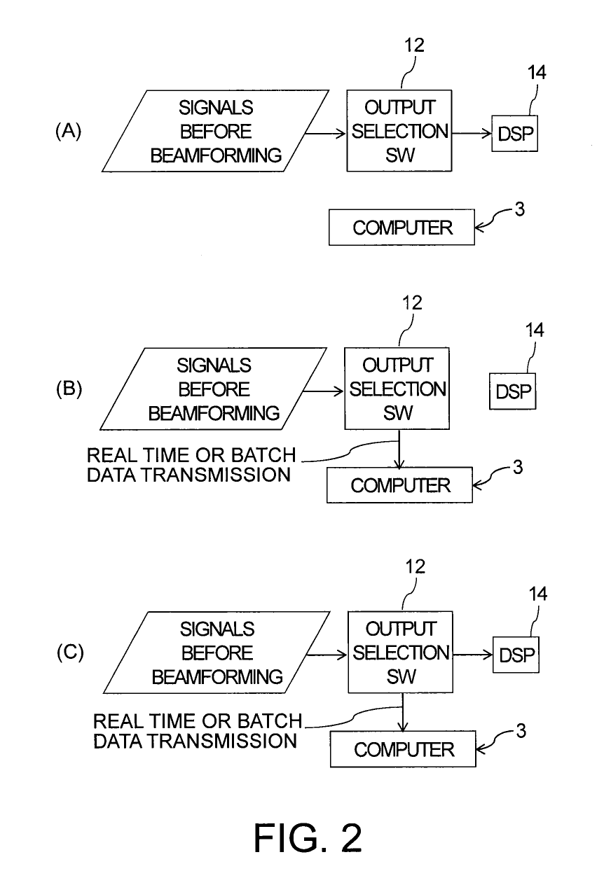

FIG. 2 is a diagram to show switching states of the reception signal output by the output selection SW 12 shown in FIG. 1.

In FIG. 2, (A) shows a state that the output of the reception signals is the DSP 14 side, (B) shows a state that the output of the reception signals is the computer 3 side and (C) shows a state that the output of the reception signals is both the DSP 14 side and the computer 3 side respectively.

To the DSP 14 side, the reception signals read from the buffer memory 11 are output in real time. Note that, the reception signals may be able to be output to the DSP 14 side posteriori by a batch data transmission. On the other hand, to the computer 3 side, the reception signals read from the buffer memory 11 can be output in real time and also output posteriori by a batch data transmission. Therefore, the beam forming processing can be performed as real time processing or batch processing in the beam forming processing part or the beam forming processing parts in either one or both of the DSP 14 and the computer 3.

As described above, one or both of the beam forming processing part of the DSP 14 and the computer 3 as an external terminal can be selected as the output or the outputs of the multiple reception signals before beam forming by switching the output selection SW 12. Additionally, multiple reception signals can be output to the computer 3 as the external terminal with switching between a real time data transmission and a batch data transmission by switching operation of the output selection SW 12.

The DSP 14 has a function to generate the first ultrasonic image data in real time by signal processing including pulse compression to multiple reception signals, before beam forming, corresponding to multiple reception channels beam forming to the multiple reception signals after the pulse compression. The DSP 14 also has a function to display the first ultrasonic image on the display 15 in real time by outputting the generated first ultrasonic image data to the display 15.

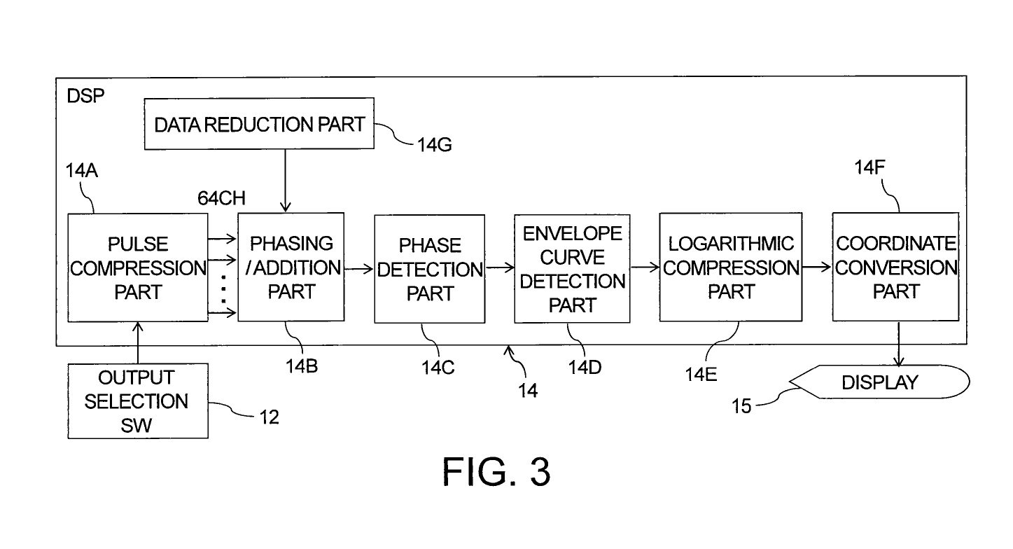

FIG. 3 is a functional block diagram of the DSP 14 shown in FIG. 1.

As shown in FIG. 3, the DSP 14 functions as a pulse compression part 14A, a phasing/addition part 14B, a phase detection part 14C, an envelope curve detection part 14D, a logarithmic compression part 14E, a coordinate conversion part 14F and a data reduction part 14G by reading and performing a data processing program.

The pulse compression part 14A has a function to perform a pulse compression processing required to multiple reception signals before beam forming when a chirp waveform having a long wave length has been used as the transmission signal.

The phasing/addition part 14B has a function to perform beam forming of the reception signals by phasing and adding multiple reception signals, after the pulse compression, corresponding to the multiple reception channels. Specifically, the phasing/addition part 14B has a function to generate ultrasonic reception data at scanning positions in an object by giving reception delay times, for the respective reception channels, to the respective reception signals and adding the reception channels.

The phase detection part 14C, the envelope curve detection part 14D, the logarithmic compression part 14E and the coordinate conversion part 14F have functions to perform known phase detection processing, envelope curve detection processing, logarithmic compression processing and coordinate conversion processing required for generating the first ultrasonic image data based on the ultrasonic reception data alter the beam forming respectively. Then, the first ultrasonic image data converted from a coordinate system of a scan format to a coordinate system of a television format is output to the display 15 from the coordinate conversion part 14F.

As described above, the phasing/addition part 14B in the DSP 14 functions as the beam forming processing part which performs the first beam forming to multiple reception signals corresponding to the multiple ultrasonic transducers 8. Thus, the phasing/addition part 14B which functions as the beam forming processing part of the DSP 14 can be selected as the output of multiple reception signals before the beam forming by the output selection SW 12. Meanwhile, the phase detection part 14C, the envelope curve detection part 14D, the logarithmic compression part 14E and the coordinate conversion part 14F in the DSP 14 have functions to generate ultrasonic image data based on the multiple reception signals subjected to the first beam forming.

The data reduction part 14G has a function to reduce the reception signals used for generating the first ultrasonic image data. Methods of reducing the reception signals include a method to decimate the reception channels to be a target of phasing and addition and a method to lower the frame rate of the first ultrasonic images displayed on the display 15 of the mobile ultrasonic diagnostic apparatus 2.

Therefore, the data reduction part 14G can reduce the reception signals used for generating the first ultrasonic image data by providing at least one of the reception channels, to be the target of phasing and addition, and the frame rate to the phasing/addition part 14B as the phasing/addition condition information as shown in FIG. 3. Note that, the frame rate may be reduced in a post-circuit of the phasing/addition part 14B by control of the data reduction part 14G.

Specifically, the reception signals used for generating the first ultrasonic image data can be reduced by the data reduction part 14G controlling the target circuit so that the first ultrasonic image data is generated with decimating at least one of the reception channels of the multiple reception signals and the frame rate.

On the other hand, the data compression circuit 16 to be the output from the output selection SW 12 has a function to perform data compression processing of the multiple reception signals, before the beam forming, output from the buffer memory 11 through the output selection SW 12. The data compression circuit 16 also has a function to output the multiple reception signals after the data compression to the computer 3 by the transmission cable 4 through the input/output I/F 17. Further, when ultrasonic image data subjected to the data compression has been received from the input/output I/F 17, the data compression circuit 16 is configured to perform data uncompressing processing of the received ultrasonic image data to output the uncompressed ultrasonic image data to the display 15.

The input/output I/F 17 of the mobile ultrasonic diagnostic apparatus 2 is an element for data exchange with the computer 3 via the transmission cable 4. Especially, the input/output I/F 17 functions as an output unit, of the mobile ultrasonic diagnostic apparatus 2, which outputs the multiple reception signals before the beam forming to the computer 3. Further, the input/output I/F 17 functions as the output unit, of the mobile ultrasonic diagnostic apparatus 2, which performs data compression of multiple reception signals to output the compressed reception signals by collaborating with the data compression circuit 16.

As the transmission cable 4, a standardized communication protocol such as Universal Serial Bus (USB) can be used. The USB3.0 which is one of USB versions can perform a data transmission at 5 G [bps] ([bit/s]).

On the other hand, when the number of the reception channels is 64 [CH], a data size of each reception signal generated in the A/D converter 10 is 10 [bit] and a frequency of each reception signal is 40 [MHz], it is required to perform a data communication at 64 [CH]×10 [bit]×40 [MHz]×25 [Gbps] for a real time communication. However, reversible differential compression processing of the multiple reception signals for the respective reception channels makes it possible to compress their data size to less than one-third since the reception signals are similar between adjacent reception channels. Therefore, a data transmission rate required for a real time communication is only 8.3 [Gbps] by the data compression.

Hence, connecting the mobile ultrasonic diagnostic apparatus 2 with the computer 3 by two USB 3.0 transmission cables 4 each allowing a data transmission at 5 [Gbps] achieves a data transmission rate of 10 [bps], and therefore, it becomes possible to transmit the reception signals acquired by the mobile ultrasonic diagnostic apparatus 2 to the computer 3 in real time.

The computer 3 has an input/output I/F 18, a calculation unit 19, an input device 20, a display unit 21 and a storage unit 22. The calculation unit 19 of the computer 3 functions as a pulse compression part 23, a phasing/addition part 24, a phase detection part 25, an envelope curve detection part 26, a logarithmic compression part 27, a coordinate conversion part 28, a data compression part 29 and a delay time correction part 30 by installing and performing a data processing program. Further, the computer 3 can store various data, generated by the calculation unit 19, in the storage unit 22 and read data from the storage unit 22 as well as inputting information to the calculation unit 19 by operation of the input device 20.

As the computer 3A, a general purpose computer such as a personal computer (PC) or a workstation can be used. Alternatively, a system consisting of mutually connected computers, so that distributed processing can be performed, may be used as the computer 3. The data processing program installed in the computer 3 can be recorded in an information recording media and distributed as a program product. Alternatively, the data processing program can be downloaded to the computer 3 using a network such as the internet.

A simple general purpose computer such as a PC can be placed adjacent to the mobile ultrasonic diagnostic apparatus 2 by connecting the computer with the mobile ultrasonic diagnostic apparatus 2 using the transmission cable 4 such as the USB. Further, the computer 3 itself may be a mobile terminal. On the contrary, a computer, such as a workstation or a system consisting of computers for distributed processing, which can perform advanced data processing can be connected to the mobile ultrasonic diagnostic apparatus 2 with relaying another computer by a hospital network.

The input/output I/F 18 of the computer 3 has a function as a data reception unit to receive multiple reception signals, before beam forming, corresponding to the multiple ultrasonic transducers 8, acquired by transmitting and receiving ultrasonic waves to and from an object with the ultrasonic transducers 8, from the mobile ultrasonic diagnostic apparatus 2 via the transmission cable 4. Additionally, the input/output I/F 18 also has a function as an image data output unit to transmit ultrasonic image data generated in the computer 3 to the mobile ultrasonic diagnostic apparatus 2 via the transmission cable 4.

The data compression part 29 of the computer 3 has a function to uncompress compressed data acquired from the input/output I/F 18 and provide the uncompressed data to the pulse compression part 23. The data compression part 29 also has a function to perform data compression of ultrasonic image data acquired from the coordinate conversion part 28 and transmit the compressed ultrasonic image data to the mobile ultrasonic diagnostic apparatus 2 via the input/output I/F 18 and the transmission cable 4.

The pulse compression part 23, the phasing/addition part 24, the phase detection part 25, the envelope curve detection part 26, the logarithmic compression part 27 and the coordinate conversion part 28 of the computer 3 have functions similar to those of the pulse compression part 14A, the phasing/addition part 14B, the phase detection part 14C, the envelope curve detection part 14D, the logarithmic compression part 14E and the coordinate conversion part 14F of the DSP 14 built in the mobile ultrasonic diagnostic apparatus 2 respectively. Specifically, the computer 3 has a function to generate the second ultrasonic image data by signal processing for generating ultrasonic image data including the pulse compression and the beam forming, similar to the DSP 14.

However, the computer 3 does not reduce reception signals for generating ultrasonic diagnostic image data. Therefore, the computer 3 functions as a data generation unit to perform pulse compression of reception signals, before the first beam forming, output from the input/output I/F 17 of the mobile ultrasonic diagnostic apparatus 2 and the second beam forming of the reception signals after the pulse compression to generate the second ultrasonic image data, having a data size larger than that of the first ultrasonic image data generated in the mobile ultrasonic diagnostic apparatus 2, based on the reception signals subjected to the second beam forming.

Then, the computer 3, in which a Central Processing Unit (CPU) and a Graphical Processing Unit (GPU) capable of data processing described above in real time are mounted, is used for the ultrasonic diagnostic system 1.

The delay time correction part 30 can be provided as required. The delay time correction part 30 has a function to control the phasing/addition part 24 so that the optimum ultrasonic reception beam can be generated by an adaptive beam forming based on the reception signals corresponding to the reception channels. More specifically, the delay time correction part 30 is configured to correct reception delay times provided to the reception signals in the phasing/addition part 24 so that the side lobe of the reception signals becomes minimum while the main lobe becomes maximum.

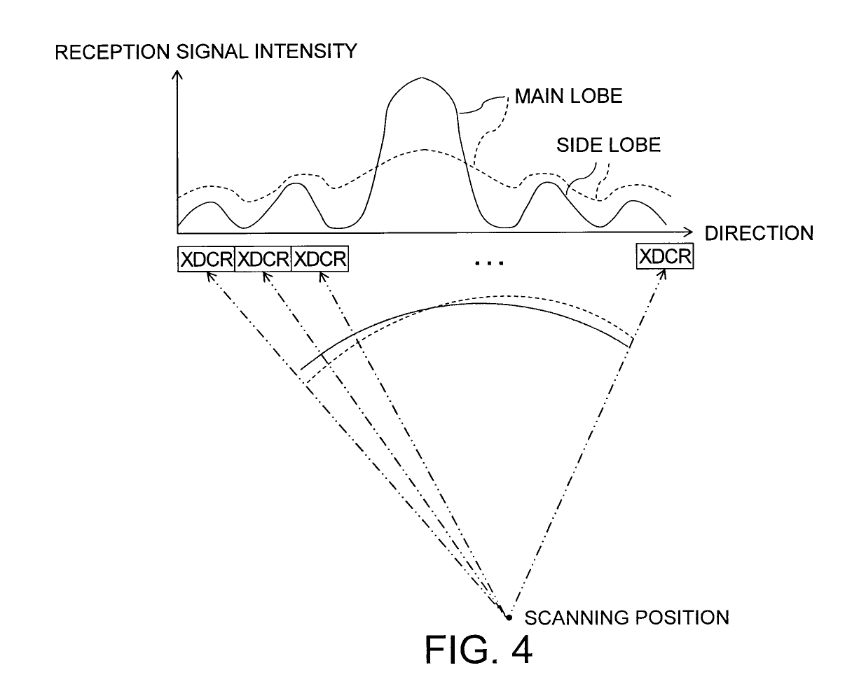

FIG. 4 is a diagram describing a correction method of the reception delay times in the delay time correction part 30 shown in FIG. 1.

In the graph in FIG. 4, the abscissa axis indicates a reception direction of an ultrasonic reflected signal and the ordinate axis indicates an intensity of a reception signal received from each reception direction. Further, a bottom part of FIG. 4 shows that an ultrasonic reception beam is formed by receiving ultrasonic reflected signals generated from a scanning position in an object with the ultrasonic transducers 8 at different timings.

Specifically, a wave front of the ultrasonic reception beam can be formed by giving appropriate reception delays to the respective reception signals in the phasing/addition part 24. Then, reception signals showing directionality can be acquired from the respective directions.

However, the sonic velocity is not uniform practically due to tissues consisting of mutually different compositions in an object. Therefore, an accurate ultrasonic reception beam from a scanning position cannot be formed when reception delays are given to the reception signals assuming a transmitting velocity of ultrasonic reflected signals is constant in an object. For example, an error occurs in a scanning position as shown by the dotted line of FIG. 4.

When intensities of reception signals, having such an error, corresponding to respective directions are plotted, the side lobe does not become small sufficiently as shown by the dotted line of the graph. Accordingly, an optimization processing for changing respective delay times of reception signals can be performed with setting the respective reception delay times given to the reception signals as parameters so that the side lobe becomes minimum while the main lobe becomes maximum. This makes it possible to obtain an ideal wave front, main lobe and side lobe of the ultrasonic reception beam as shown by the solid line of FIG. 4.

Note that, the adaptive beam forming performed by the delay time correction part 30 requires a very large data processing amount. Accordingly, the delay time correction part 30 is provided when the computer 3 is a workstation having a large data processing capacity on the like. Therefore, a medical image processing apparatus may be used as the computer 3 for the ultrasonic diagnostic system 1. Further, the adaptive beam forming is generally performed when the second ultrasonic image is not displayed in real time, i.e., the second ultrasonic image is displayed on the display unit 21 after an ultrasonic scan.

Next, the operation and the action of the ultrasonic diagnostic system 1 will be described.

First, the output selection SW 12 is operated by handling of the control panel 13 to select an output of reception signals. Here, a description will be given for an example case of selecting the DSP 14 and the computer 3 as the outputs. After determining the output, the ultrasonic probe formed at the end of the mobile ultrasonic diagnostic apparatus 2 is put to a diagnostic part of an object.

Next, transmission signals, such as chirp waves, to which a pulse compression can be performed, are applied to the respective ultrasonic transducers 8 with delay times for the transmission beam forming from the transmission circuit 5 via the transmission/reception separation circuit 6 and the high pressure SW 7. Therefore, the ultrasonic signals are transmitted to a scanning position of the object from the respective ultrasonic transducers 8. Consequently, the ultrasonic reflected signals generated at the scanning position are received by the respective ultrasonic transducers 8. The received ultrasonic reflected signals are converted to electric reception signals in the corresponding ultrasonic transducers 8 to be output.

The multiple reception signals output from the ultrasonic transducers 8 are output to the amplifier 9 through corresponding reception channels via the high pressure SW 7 and the transmission/reception separation circuit 6. The reception signals for the reception channels amplified in the amplifier 9 are converted to digital signals in the A/D converter 10 and stored in the buffer memory 11.

The reception signals corresponding to the ultrasonic transducers 8 and the reception channels are output to the DSP 14 and the data compression circuit 16 from the buffer memory 11 through the output selection SW 12 in real time.

In the DSP 14, a signal processing for generating the first ultrasonic image data is performed. Specifically, a pulse compression for the reception signals is performed in the pulse compression part 14A. Next, an ultrasonic reception beam is formed by phasing and addition of the reception signals in the phasing/addition part 14B.

However, it might become difficult to generate the first ultrasonic image data in real time with the data processing capacity of the DSP 14. In that case, decimation processing of the reception signals corresponding to specific reception channels and/or specific time phases is performed by the data reduction part 14G.

Specifically, the reception channels can be decimated by sub array processing which performs phase correction and addition of the reception signals every multiple channels. That is, the data processing amount in the DSP 14 can be reduced by reducing the pixel number of the first ultrasonic image data to be a target of real time display in the mobile ultrasonic diagnostic apparatus 2.

Additionally, a frame rate of the first ultrasonic image data to be a target of real time display in the mobile ultrasonic diagnostic apparatus 2 can be lower than a frame rate in an actual ultrasonic scan by adding the reception signals every multiple time phases. Specifically, the data processing amount in the DSP 14 can be also reduced by lowering the frame rate of the first ultrasonic image data.

For example, the phasing/addition processing can be performed at a rate of one time per 8 times of acquisition of reception signals for 1 frame. In this case, when a frame rate of an ultrasonic scan is 32 [fps] ([frame/s]), the frame rate of the first ultrasonic image data becomes 4 [fps]. Additionally, if the reception signals are subjected to the phasing and addition every 2 channels, a load of the phasing/addition processing in the DSP 14 can be reduced to ½×⅛= 1/16.

The degree in decimation of the reception channels and the frames like this can be set variably depending on the data processing amount in the DSP 14 and the data processing rate of the DSP 14. Further, the pulse compression may not be performed for reducing the data processing amount in the DSP 14.

The first ultrasonic image displayed on the display 15 of the mobile ultrasonic diagnostic apparatus 2 is referred as an image for confirming a scan part and is not used for diagnosis. Therefore, the number of the addition of the reception channels and the frame rate can be adjusted so that the first ultrasonic image can be displayed on the mobile ultrasonic diagnostic apparatus 2 in real time with at least an image quality required for performing an ultrasonic scan. For example, the pixel number and the frame rate of the first ultrasonic image can be set to approximately 256×256 and 2 [Hz] respectively. Next, a phase detection processing, an envelope curve detection processing, a logarithmic compression processing and a coordinate conversion processing are performed for the reception data after the phasing and addition by the phase detection part 14C, the envelope curve detection part 14D, the logarithmic compression part 14E and the coordinate conversion part 14F respectively. Consequently, the first ultrasonic image data is generated. The generated first ultrasonic image data is output to the display 15. Therefore, a user can adjust a position and a direction of the ultrasonic probe formed in the mobile ultrasonic diagnostic apparatus 2 with confirming a scanning part of the ultrasonic scan.

On the other hand, the second ultrasonic image used for actual diagnosis is generated and displayed in real time by signal processing in the computer 3. For that purpose, the reception signals, before the beam forming, output from the input/output I/F 17 through the output selection SW 12 and the data compression circuit 16 from the buffer memory 11 of the mobile ultrasonic diagnostic apparatus 2 is transmitted to the computer 3 as compressed data via the transmission cable 4.

Consequently, the compressed data of the reception signals corresponding to the ultrasonic transducers 8 and the reception channels is given to the data compression part 29 via the input/output I/F 18 in the computer 3. Then, the data compression part 29 performs uncompressing processing of the compressed data to acquire uncompressed data of the reception signals corresponding to the ultrasonic transducers 8 and the reception channels.

Next, the pulse compression of the reception signals, the beam forming by the phasing and addition, the phase detection processing of the reception data after the beam forming, the envelope curve detection processing, the logarithmic compression processing and the coordinate conversion processing are performed in the pulse compression part 23, the phasing/addition part 24, the phase detection part 25, the envelope curve detection part 26, the logarithmic compression part 27 and the coordinate conversion part 28 of the computer 3 respectively. Consequently, the second ultrasonic image data, of which pixel number is approximately 512×512 and frame rate is approximately 60 [Hz], having an image quality equivalent to that of a high specification apparatus can be generated in the computer 3 for example.

Then, the generated second ultrasonic image is displayed on the display unit 21 in real time. Therefore, a user can diagnose the scan part of the object by observing the second ultrasonic image.

Further, the second ultrasonic image data can be also transmitted and displayed to and on the mobile ultrasonic diagnostic apparatus 2. In that case, the second ultrasonic image data is provided to the data compression part 29 from the coordinate conversion part 28. Then, the second ultrasonic image data compressed in the data compression part 29 is transferred to the mobile ultrasonic diagnostic apparatus 2 via the input/output I/F 18 of the computer 3 and the transmission cable 4.

Subsequently, the compressed data of the second ultrasonic image data is input to the data compression circuit 16 via the input/output I/F 17 in the mobile ultrasonic diagnostic apparatus 2. Then, the uncompressed second ultrasonic image data after uncompressing processing in the data compression circuit 16 is output to the display 15. Therefore, a user can diagnose the object by observing the second ultrasonic image displayed on the display 15 of the mobile ultrasonic diagnostic apparatus 2.

Additionally, the adaptive beam forming with the optimization of the delay times for the reception signals can be performed after the scan by controlling the phasing/addition part 24 by the delay time correction part 30 in the computer 3. In that case, the compressed data or the uncompressed data of the reception signals is stored in the storage unit 22 of the computer 3. Then, the uncompressed data of the reception signals is provided to the pulse compression part 23.

Next, the second ultrasonic image data with an improved image quality, which is difficult to be acquired even by a conventional high specification apparatus, can be generated by signal processing including the adaptive beam forming based on the reception signals after the pulse compression. The generated second ultrasonic image data can be displayed on the display unit 21 of the computer 3 or the display 15 of the mobile ultrasonic diagnostic apparatus 2.

Note that, the transmission of the reception signals before the beam forming to the computer 3 side can be also performed not in real time but later. In that case, the computer 3 side is selected as the output of the output selection SW 12 after the scan. Then, the reception signals, before the beam forming, read from the buffer memory 11 are output to the computer 3 side by batch data transmission. In this case, the adaptive beam forming can be also performed as an option.

That is, the ultrasonic diagnostic system 1 as described above is a system configured to be able to apply transmission signals such as chirp waves, to which pulse compression can be performed, to the ultrasonic transducer 8 included in the mobile ultrasonic diagnostic apparatus 2. Additionally, the ultrasonic diagnostic system 1 can perform signal processing for generating an ultrasonic image for diagnosis after the pulse compression in real time and in parallel in the computer 3 other than the mobile ultrasonic diagnostic apparatus 2 in order to solve a problem that pulse compression circuits for the number of reception channels are required for pulse compression of the reception signals.

Therefore, the ultrasonic diagnostic system 1 can make the size of the mobile ultrasonic diagnostic apparatus 2 smaller without reducing the numbers of the ultrasonic transducers 8 and the channels by integration of circuits in the transmission system. Further, the production cost and the price of the mobile ultrasonic diagnostic apparatus 2 can be reduced. On the other hand, the second ultrasonic image having an image quality equivalent to or more than that of a high specification apparatus can be displayed on the computer 3 or the mobile ultrasonic diagnostic apparatus 2.

### Second Embodiment

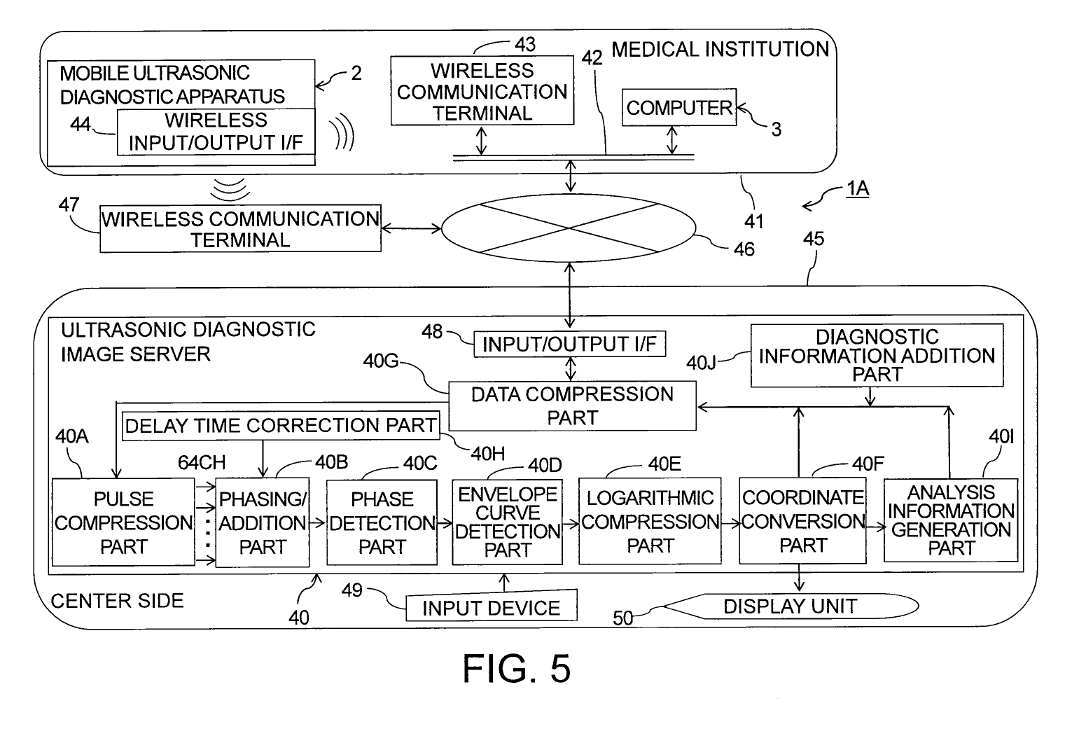

FIG. 5 is a block diagram showing an ultrasonic diagnostic system according to the second embodiment of the present invention.

An ultrasonic diagnostic system 1A shown in FIG. 5 is different from the ultrasonic diagnostic system 1 in the first embodiment shown in FIG. 1 in the point that the mobile ultrasonic diagnostic apparatus 2 is connected with an ultrasonic diagnostic image server 40 placed in a remote location via a network. Other structures and operations are substantially same as those of the ultrasonic diagnostic system 1 shown in FIG. 1. Therefore, the same reference numbers are used for same elements as those in FIG. 1, and the description thereof is omitted.

The ultrasonic diagnostic system 1A has the mobile ultrasonic diagnostic apparatus 2, the computer 3 and the ultrasonic diagnostic image server 40. The mobile ultrasonic diagnostic apparatus 2 and the computer 3 are placed in a medical institution 41 such as a medical clinic. In the medical institution 41, a local area network (LAN) 42 is laid. To the LAN 42, each of the computer 3 and a wireless communication terminal 43 is connected.

Further, the mobile ultrasonic diagnostic apparatus 2 includes a wireless input/output I/F 44. Then, the mobile ultrasonic diagnostic apparatus 2 is connected with the LAN 42 in the medical institution 41 by wireless communication between the wireless input/output I/F 44 and the wireless communication terminal 43. Specifically, the mobile ultrasonic diagnostic apparatus 2 can perform data communication with the computer 3.

On the other hand, the ultrasonic diagnostic image server 40 is placed in the center 45 side such as a large medical institution which generates and provides ultrasonic image data. The ultrasonic diagnostic image server 40 is connected with the LAN 42 in the medical institution 41, in which the mobile ultrasonic diagnostic apparatus 2 is equipped, via a wide area network 46 such as internet or a dedicated line. Further, the wireless communication terminal 47 is connected with the wide area network 46.

Therefore, the mobile ultrasonic diagnostic apparatus 2 is connected with the ultrasonic diagnostic image server 40 via the wireless communication terminal 43 connected with the LAN 42 or the wireless communication terminal 47 connected with the wide area network 46. Further, the computer 3 is connected with the ultrasonic diagnostic image server 40 via the LAN 42 and the wide area network 46. Specifically, the ultrasonic diagnostic image server 40 is connected with each of the mobile ultrasonic diagnostic apparatus 2 and the computer 3 via the network.

Then, the reception signals, corresponding to the ultrasonic transducers 8 and the reception channels, before the beam forming can be transferred to the ultrasonic diagnostic image server 40 from the wireless input/output I/F 44 in the mobile ultrasonic diagnostic apparatus 2 by wireless communication. Specifically, the wireless input/output I/F 44 in the mobile ultrasonic diagnostic apparatus 2 functions as an output unit which transmits the reception signals before the beam forming to the ultrasonic diagnostic image server 40.

For example, when the IEEE 802.11 n which is a standard of the wireless LAN specified by IEEE (The Institute of Electrical and Electronics Engineers, Inc.) is used for the wireless communication, reception signals can be transferred from the mobile ultrasonic diagnostic apparatus 2 wirelessly by a data transfer rate of 600 [Mbps]. In this case, all the reception signals cannot be transferred to the ultrasonic diagnostic image server 40 from the mobile ultrasonic diagnostic apparatus 2 in real time. Accordingly, the reception signals are transferred to the ultrasonic diagnostic image server 40 sequentially during an ultrasonic scan. Alternatively, all the reception signals are stored in the buffer memory 11 of the mobile ultrasonic diagnostic apparatus 2 once and the reception signals are transferred to the ultrasonic diagnostic image server 40 sequentially after the ultrasonic scan with the batch data transmission form by switching the output selection SW 12.

The ultrasonic diagnostic image server 40 includes an input/output I/F 48. The input/output I/F 48 is connected with the wide area network 46. Therefore, the input/output I/F 48 functions as a data reception unit of the ultrasonic diagnostic image server 40 which receives reception signals, before the beam forming, corresponding to the ultrasonic transducers 8, acquired by transmitting and receiving ultrasonic waves to and from an object with the ultrasonic transducers 8, from the mobile ultrasonic diagnostic apparatus 2 via a network.

The ultrasonic diagnostic image server 40 is configured by a computer, capable of a large scale data processing, which functions as the pulse compression part 40A, the phasing/addition part 40B, the phase detection part 40C, the envelope curve detection part 40D, the logarithmic compression part 40E, the coordinate conversion part 40F, the data compression part 40G, the delay time correction part 40H, the analysis information generation part 40I and the diagnostic information addition part 40J by installing a data processing program on the computer to be executed. Note that, a computer to configure the ultrasonic image server 40 may be also a system consisting of mutually connected computers which can perform distributed processing.

Further, each of an input device 49 and a display unit 50 are connected with the ultrasonic diagnostic image server 40. Each of the input device 49 and the display unit 50 may be connected with the ultrasonic diagnostic image server 40 indirectly via other computers.

The pulse compression part 40A, the phasing/addition part 40B, the phase detection part 40C, the envelope curve detection part 40D, the logarithmic compression part 40E, the coordinate conversion part 40F, the data compression part 40G and the delay time correction part 40H of the ultrasonic diagnostic image server 40 have functions similar to those of the pulse compression part 23, the phasing/addition part 24, the phase detection part 25, the envelope curve detection part 26, the logarithmic compression part 27, the coordinate conversion part 28, the data compression part 29 and the delay time correction part 30 of the computer 3 shown in FIG. 1 respectively. Therefore, when it is difficult to provide the delay time correction part 30 to the computer 3 shown in FIG. 5 from the perspective of a data processing capacity, the delay time correction part 40H may be provided only to the ultrasonic diagnostic image server 40.

The ultrasonic diagnostic image server 40, having such a function as described above, functions as a data generation unit to perform the second beam forming of reception signals, before the first beam forming, output from the wireless input/output I/F 44 of the mobile ultrasonic diagnostic apparatus 2 to generate the second ultrasonic image data of which data size is larger than that of the first ultrasonic image data generated in the mobile ultrasonic diagnostic apparatus 2 based on the reception signals subjected to the second beam forming, similarly to the computer 3 shown in FIG. 1.

Specifically, signal processing, including the beam forming such as the pulse compression, the phasing/addition processing, the phase detection processing, the envelope curve detection processing, the logarithmic compression processing and the coordinate conversion processing, is performed to the reception signals in the ultrasonic diagnostic image server 40 off line. In this case, the reception signals are not reduced for generating the second ultrasonic image data in the ultrasonic diagnostic image server 40 differently from the signal processing in the mobile ultrasonic diagnostic apparatus 2. Further, it is also possible to perform the adaptive beam forming by operation of the delay time correction part 40H.

Therefore, the second ultrasonic image data having an improved image quality equivalent to or more than that of a conventional high specification apparatus can be generated. The second ultrasonic image data can be output to the display unit 50 connected with the ultrasonic diagnostic image server 40. Therefore, when the center 45 side is a large scale medical institution, diagnosis based on the second ultrasonic image can be performed by a user such as a doctor.

Further, the analysis information generation part 40I in the ultrasonic diagnostic image server 40 has a function to extract a lesion part automatically by image analysis processing such as threshold processing of the second ultrasonic image data. The analysis information generation part 40I also has a function to add area information of the extracted lesion part to the second ultrasonic image data as incidental information. Additionally, the diagnostic information addition part 40J has a function to add diagnostic information by a doctor to the second ultrasonic image data as incidental information with operating the input device 49.

Therefore, in the center 45 side, the second ultrasonic image data to which position information of a lesion part and diagnostic information are added can be generated, as required. Then, the generated second ultrasonic image data can be transmitted to an arbitrary device such as the mobile ultrasonic diagnostic apparatus 2 or the computer 3 in the medical institution 41 via the network. Specifically, the input/output I/F 48 of the ultrasonic diagnostic image server 40 functions as a data transmission unit which transmits the second ultrasonic image data to a device such as the computer 3 having the display unit 21 via the network.

Then, the second ultrasonic image can be displayed using an arbitrary monitor for observation such as the display unit 21 included in the computer 3 in the medical institution 41. Consequently, at the medical institution 41 side, diagnosis of an object can be performed by observing the second ultrasonic image. Further, position information of a lesion part and diagnostic information obtained in the center 45 side can be displayed on a monitor in the medical institution 41 side with the second ultrasonic image. Therefore, the second ultrasonic image can be displayed on a monitor in the small medical institution 41 such as a clinic with a diagnostic result obtained by observing the second ultrasonic image by a specialized doctor in the center 45 side for example.

The ultrasonic diagnostic system 1A in the second embodiment as mentioned above is a system configured to be able to perform signal processing, after pulse compression, for generating the second ultrasonic image for diagnosis, in the ultrasonic diagnostic image server 40 by connecting the mobile ultrasonic diagnostic apparatus 2 with the ultrasonic diagnostic image server 40 placed in a remote location via a network.

Therefore, an effect similar to that by the ultrasonic diagnostic system 1 in the first embodiment can be obtained by the ultrasonic diagnostic system 1A in the second embodiment. Additionally, advanced signal processing, such as the adaptive beam forming, for acquiring a higher image quality can be performed easily with a common computer. Moreover, a remote medical care can be also performed with generating the second ultrasonic image for diagnosis.

### Other Embodiments

While certain embodiments have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

For example, though an example of connecting the mobile ultrasonic diagnostic apparatus 2 with the computer 3 by the transmission cable 4 is described in the first embodiment, the mobile ultrasonic diagnostic apparatus 2 may be able to communicate with the computer 3 by wireless communication. On the contrary, in the second embodiment, the mobile ultrasonic diagnostic apparatus 2 may be also connected with each of the computer 3 and the ultrasonic diagnostic image server 40 by the transmission cable 4. Specifically, the mobile ultrasonic diagnostic apparatus 2, the computer 3 and the ultrasonic diagnostic image server 40 can be connected mutually via a wired or wireless network.

Further, the mobile ultrasonic diagnostic apparatus 2 may be various types of ultrasonic diagnostic apparatuses such as a portable standing ultrasonic diagnostic apparatus. Additionally, not only the DSP 14 but a processor and/or a circuit having an equivalent data processing function can be used for generating the first ultrasonic image data in the ultrasonic diagnostic apparatus.

What is claimed is:

1. An ultrasonic diagnostic system comprising:
a data acquiring unit configured to acquire reception signals corresponding to ultrasonic transducers by transmitting and receiving ultrasonic waves to and from an object using the ultrasonic transducers;
a beam forming processing unit configured to apply beam forming to the reception signals;
a processor configured to generate ultrasonic image data based on reception signals subjected to the beam forming; and
an output unit configured to output the reception signals before the beam forming to an outside terminal.

2. An ultrasonic diagnostic system comprising:
an ultrasonic diagnostic apparatus; and
a computer connected to said ultrasonic diagnostic apparatus through a network, wherein said ultrasonic diagnostic apparatus includes:
a data acquiring unit configured to acquire reception signals corresponding to ultrasonic transducers by transmitting and receiving ultrasonic waves to and from an object using the ultrasonic transducers;
a beam forming processing unit configured to apply a first beam forming to the reception signals;
a processor configured to generate first ultrasonic image data based on reception signals subjected to the first beam forming; and
an output unit configured to output the reception signals before the first beam forming to said computer, and
said computer functions as a data generation unit configured to apply a second beam forming to the reception signals before the first beam forming output from said output unit to generate second ultrasonic image data having a data size larger than that of the first ultrasonic image data based on the reception signals subjected to the second beam forming.

3. An ultrasonic diagnostic system comprising:
an ultrasonic diagnostic apparatus placed in a medical institution;
a computer placed in the medical institution and having a display unit; and
an ultrasonic diagnostic image server placed in a center side and connected with each of said ultrasonic diagnostic apparatus and said computer through a network,
wherein said ultrasonic diagnostic apparatus includes:
a data acquiring unit configured to acquire reception signals corresponding to ultrasonic transducers by transmitting and receiving ultrasonic waves to and from an object using the ultrasonic transducers;
a beam forming processing unit configured to apply a first beam forming to the reception signals;
a processor configured to generate first ultrasonic image data based on reception signals subjected to the first beam forming; and
an output unit configured to transmit the reception signals before the first beam forming to said ultrasonic diagnostic image server, and
said ultrasonic diagnostic image server includes:
a data generation unit configured to apply the second beam forming to the reception signals before the first beam forming output from said output unit to generate second ultrasonic image data having a data size larger than that of the first ultrasonic image data based on the reception signals subjected to the second beam forming; and
a data transmission unit configured to transmit the second ultrasonic image data to said computer.

4. An ultrasonic diagnostic system comprising:
a data reception unit configured to receive reception signals before a beam forming and corresponding to ultrasonic transducers from an ultrasonic diagnostic apparatus through a network, the reception signals being acquired by transmitting and receiving ultrasonic waves to and from an object using the ultrasonic transducers; and
a data generation unit configured to apply a beam forming to the reception signals to generate ultrasonic image data based on reception signals subjected to the beam forming.

5. An ultrasonic diagnostic system comprising:
a data reception unit configured to receive reception signals before a beam forming and corresponding to ultrasonic transducers from an ultrasonic diagnostic apparatus through a network, the reception signals being acquired by transmitting and receiving ultrasonic waves to and from an object using the ultrasonic transducers;
a data generation unit configured to apply a beam forming to the reception signals to generate ultrasonic image data based on reception signals subjected to the beam forming; and
a data transmission unit configured to transmit the ultrasonic image data to a computer having a display unit through a network.

6. An ultrasonic diagnostic system of claim 1, further comprising:
a switch configured to select one or both of said beam forming processing unit and the outside terminal as an output destination of the reception signals before the beam forming.

7. An ultrasonic diagnostic system of claim 6,
wherein said switch is configured to be able to switch a real time data transmission and a batch data transmission to output the reception signals before the beam forming.

8. An ultrasonic diagnostic system of claim 1,
wherein the ultrasonic image data is generated by signal processing including pulse compression of the reception signals before the beam forming.

9. An ultrasonic diagnostic system of claim 2,
wherein said data generation unit is configured to perform processing for changing respective delay times of the reception signals before the beam forming so as to maximize a main lobe and minimize a side lobe.

10. An ultrasonic diagnostic system of claim 1,
wherein said processor is configured to generate the ultrasonic image data with decimating at least one of reception channels of the reception signals subjected to the beam forming and a frame rate.

11. An ultrasonic diagnostic system of claim 1,
wherein said output unit is configured to perform a data compression of the reception signals before the beam forming to output the reception signals before the beam forming.

12. An ultrasonic diagnostic system of claim 4,
wherein said data generation unit consists of a computer system performing distributed processing with mutually connected computers.

13. An ultrasonic diagnostic method comprising:
acquiring reception signals corresponding to ultrasonic transducers by transmitting and receiving ultrasonic waves to and from an object using the ultrasonic transducers;
applying beam forming to the reception signals;
generating ultrasonic image data with a processor based on reception signals subjected to the beam forming; and
outputting the reception signals before the beam forming to an outside terminal.
